(19) United States
(12) Patent Application Publication
(10) Pub. No.: US 2026/0294774 A1
(43) Pub. Date: Oct. 1, 2026

(54) FERMENTED BINDING SERUM

(71) Applicant: ACTERA INGREDIENTS, INC., Newtown, PA (US)

(72) Inventor: Daniel Winn, Princeton, NJ (US)

(73) Assignee: ACTERA INGREDIENTS, INC., Newtown, PA (US)

(21) Appl. No.: 19/479,135

(22) PCT Filed: Apr. 26, 2024
(86) PCT No.: PCT/US2024/026629

§ 371 Date: Oct. 27, 2025

Related U.S. Application Data
(60) Provisional application No. 63/498,920, filed on Apr. 28, 2023.
(60) Provisional application No. PCT/US23/29133, filed on Jul. 31, 2023.

(51) Int. Cl. A61K 8/65; A61K 8/60; A61K 8/99; A61Q 5/02; A61Q 5/12

(52) U.S. Cl. CPC A61K 8/65; A61K 8/60; A61K 8/99; A61Q 5/02; A61Q 5/12; A61K 2800/85

(57) ABSTRACT

The present disclosure is generally directed to a keratin fermentation broth or a fraction or an isolate thereof for use in cosmetic compositions, particularly hair treatment compositions such as shampoos and conditioners. The keratin fermentation broth comprises fermented a) keratin and/or hydrolyzed keratin, b) a Lactobacillus species, and c) a simple sugar.

### CROSS-REFERENCE TO RELATED APPLICATIONS

This application is a continuation-in-part of and claims priority to International Application No. PCT/US2023/029133 filed on Jul. 31, 2023, and claims priority to U.S. Provisional Application No. 63/498,920 filed on Apr. 28, 2023, each of which is incorporated by reference herein in its entirely.

### TECHNICAL FIELD

The present disclosure is generally directed to a keratin fermentation broth for use in cosmetic compositions, particularly hair treatment compositions such as shampoos and conditioners.

### BACKGROUND

Hair is formed from layers of keratin protein which are polymeric. Damage to hair can occur as hair ages, especially from environmental factors such as UV light, ozone, and moisture. Damage can also occur through various treatments of hair, including physical (e.g. heat from straightening or curling) and chemical (e.g. coloring and hair relaxers).

Keratin-containing cosmetic formulations such as shampoos are thought to strengthen hair by fortifying and repairing keratin structure. (See WO 1995/017157 A2, WO 2004/047774 A1, and WO 2012/025615 A2 for examples of keratin-containing cosmetic formulations.) However, no formulations have been reported that comprise a keratin fermentation broth.

### SUMMARY

The present disclosure provides a keratin fermentation broth comprising a) keratin and/or hydrolyzed keratin, b) a Lactobacillus species, and c) a simple sugar, or a fraction or an isolate thereof. It is further directed to a cosmetic composition comprising the keratin fermentation broth.

The present disclosure is also directed to a method of treating hair fibers, the method comprising contacting the hair fibers with a hair treatment composition comprising a keratin fermentation broth comprising a) keratin and/or hydrolyzed keratin, b) Lactobacillus acidophilus, and c) a simple sugar, or a fraction or an isolate thereof.

The present disclosure is further directed to a method of producing a keratin fermentation broth, the method comprising:
a) combining keratin and/or hydrolyzed keratin, Lactobacillus acidophilus, and a simple sugar, in an aqueous fermentation solution; and
b) fermenting the aqueous fermentation solution at a temperature of from about 2 to about 53° C. and pH of from about 4.5 to about 6.5 to make the keratin fermentation broth.

The present disclosure is further directed a keratin fermentation product produced by exposing keratin and/or hydrolyzed keratin to fermentation of a simple sugar with a Lactobacillus species.

The present disclosure is also directed to a product comprising keratin and/or hydrolyzed keratin exposed to fermentation of a simple sugar with a Lactobacillus species.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 depicts the mean contact angle for 20% SLES and Keratin Fermentation Broth Simple Solution 0.1% treatments in Example 5.

FIG. 2 depicts the mean remaining time for 20% SLES and Keratin Fermentation Broth Simple Solution 0.1% treatments in Example 5.

FIG. 3 depicts the mean breakage for 20% SLES and Keratin Fermentation Broth Simple Solution 0.1% treatments in Example 6.

FIG. 4 depicts the mean values of the water vaporization enthalpy for 20% SLES and Keratin Fermentation Broth Simple Solution 0.1% treatments for integrity in Example 7.

FIG. 5 depicts the DSC curve with enthalpy of keratin denaturation for the 20% SLES (control) treatment in Example 7.

FIG. 6 depicts the DSC curve with enthalpy of keratin denaturation for the Keratin Fermentation Broth Simple Solution 0.1% treatment in Example 7.

FIG. 7 depicts mean luster values for CTRP, CTRN, and Keratin Fermentation Broth Simple Solution 0.1% treatments in Example 8.

FIG. 8A is a fluorescence microscopy image of cross-sections of relaxed MB hair soaked in 0.1% dye-only control (after purification) for 24 hours depicting natural hair fluorescence in the green channel in Example 9.

FIG. 8B is a fluorescence microscopy image of cross-sections of relaxed MB hair soaked in 0.1% dye-only control (after purification) for 24 hours depicting no presence of dye in the red channel in Example 9.

FIG. 9A is a fluorescence microscopy image of cross-sections of relaxed MB hair soaked in 0.1% labeled Fermented Binding Serum for 24 hours depicting natural hair fluorescence in the green channel in Example 9.

FIG. 9B is a fluorescence microscopy image of cross-sections of relaxed MB hair soaked in 0.1% labeled Fermented Binding Serum for 24 hours in the red channel in Example 9. Visible penetration of Fermented Binding Serum into the cortex was detected.

FIG. 10A is a fluorescence microscopy image of cross-sections of relaxed MB hair soaked in 0.1% labeled Keratec™ IFP PE (Croda Inc.) for 24 hours depicting natural hair fluorescence in the green channel in Example 9.

FIG. 10B is a fluorescence microscopy image of cross-sections of relaxed MB hair soaked in 0.1% labeled Keratec™ IFP PE (Croda Inc.) for 24 hours in the red channel in Example 9. Keratec™ is concentrated in the cuticle with minimal external cortex penetration in some fibers.

FIG. 11 depicts mean corrected total fluorescence from penetration inside of hair fibers by the fluorescence labeled peptides after 24 hours of treatment in Example 9. The asterisk (*) indicates statistical significance (p<0.05).

FIG. 12 depicts odor intensity of unfermented hydrolyzed keratin control and fermented binding serum (n=18, 6 panelists×3 evaluations).

FIG. 13 depicts hedonic tone of unfermented hydrolyzed keratin control and fermented binding serum (n=18, 6 panelists×3 evaluations).

FIG. 14 depicts a spider chart representing a general profile and sensory comparisons of unfermented hydrolyzed keratin control and fermented binding serum.

FIG. 15 depicts a graphical comparison of the concentrations (μg/m³) of each chemical group for the unfermented hydrolyzed keratin control and fermented binding serum.

### DETAILED DESCRIPTION

### Keratin Fermentation Broths

This disclosure describes a keratin fermentation broth comprising a) keratin and/or hydrolyzed keratin, b) a Lactobacillus species, and c) a simple sugar, or a fraction or an isolate thereof.

Specifically, the keratin fermentation broth can be referred to by its International Nomenclature Cosmetic Ingredient (INCI) name Lactobacillus/Honey/Keratin Ferment, mono ID 37496.

The keratin and/or hydrolyzed keratins can be extracted from wool. Hydrolyzed keratin is more water-soluble than unhydrolyzed keratin. Many methods of hydrolyzing keratin are known, including those disclosed in WO 2010/114938 A1 by KERAPLAST TECHNOLOGIES, LTD, which is incorporated herein by reference.

In various embodiments, the fermentation organism is Lactobacillus acidophilus.

The carbohydrate source for fermentation can be or comprise any simple sugar suitable for Lactobacillus fermentation. In at least one embodiment, the simple sugar is provided by honey, e.g., the carbohydrate source for fermentation is provided by honey.

Lactobacillus fermentation has been used for thousands of years to transform and preserve food products, but it has not yet been applied to animal keratins. It was surprisingly found that improved conditioning, hair treatments, hair styling, combing and other properties were achieved by fermenting wool keratins. Honey contains enzymes (such as diastase, invertase, glucose oxidase, catalase, glucosylceramidase, α-amylase, α-glucosidase, β-glucosidase, and proteases) that may have unknown impact on the keratin structures and/or impact on the fermentation process, and may contribute to the resulting benefits. It was also surprisingly found that the hydrolyzed keratin fermentation broth has a reduced or improved odor compared to unfermented hydrolyzed keratin.

In various embodiments, the keratin fermentation broth comprises an aqueous fermentation broth.

Keratin fermentation broths are typically acidic. Lactobacillus fermentation generally acts on a carbohydrate to produce lactic acid. A basic amino acid and/or amino alcohol, accordingly, can serve to adjust the pH to a suitable level (i.e., less acidic).

In various embodiments, the keratin fermentation broths described herein can have a pH of about 7 or less, such as about 6.5 or less, about 6 or less, about 5.5 or less, about 5 or less, about 4.5 or less, about 4 or less, or about 3 or less. In some embodiments, the keratin fermentation broths described herein can have a pH of from about 3 to about 7, from about 3 to about 6, from about 3 to about 5, from about 3 to about 4, from about 4 to about 7, from about 4 to about 6, or from about 4 to about 5.

In various embodiments, the keratin and/or hydrolyzed keratin in the keratin fermentation broth has an average molecular weight of from about 1 kDa to about 10 kDa, from about 3 kDa to about 8 kDA, or from about 4 kDa to about 5 kDa. In at least one embodiment, the keratin and/or hydrolyzed keratin has an average molecular weight of less than 10 kDa, e.g., between about 1 kDa and 10 kDa. In at least one embodiment, the keratin and/or hydrolyzed keratin has an average molecular weight of about 2.5 kDa, about 3 kDa, about 4.5 kDa, or about 5 kDa. In at least one example, the keratin and/or hydrolyzed keratin in the keratin fermentation broth has an average molecular weight greater than 1 kDa and less than or equal to 20 kDa, or greater than 1 kDa and less than or equal to 15 kDa.

### Methods of Lactobacillus Fermentation

The present disclosure is further directed to a method of producing a keratin fermentation broth, the method comprising:
a) combining keratin and/or hydrolyzed keratin, Lactobacillus acidophilus, and a simple sugar in an aqueous fermentation solution; and
b) fermenting the aqueous fermentation solution at a temperature of from about 2° C. to about 53° C. and pH of from about 4.5 to about 6.5 to produce the keratin fermentation broth.

Lactobacillus fermentation requires no special equipment. It is a traditional process used for various foods (dairy, pickling of vegetable, fermentation of sausages and meats). It has previously been used to ferment materials used in cosmetic compositions, and many methods are known in the art (See, for example, U.S. Pat. No. 9,295,704, US 2021/0052486 A1, KR 101045310 B1, KR 101452770 B1 and KR 20000039570 A). The fermentation can be carried out in almost any container or vessels at any scale, and at more or less ambient conditions. Lactobacillus fermentation generally acts on a carbohydrate to produce lactic acid.

A Lactobacillus species, simple sugar, and keratin and/or hydrolyzed keratin are combined in a vessel. In some embodiments, the Lactobacillus species is Lactobacillus acidophilus, and the simple sugar is provided by honey.

In some embodiments, the fermentation is carried out in an aqueous solution. Typically, the keratin and/or hydrolyzed keratin are rendered sufficiently soluble in the aqueous fermentation solution or medium, for example, from about 2 to about 15% by weight keratin and/or hydrolyzed keratin. Hydrolyzed keratin is more soluble in water than keratin, so a fermentation at higher concentration is possible if the keratin is first hydrolyzed. Fermentation can be carried out at a temperature of from about 2° C. to 53° C. and at a pH varying from about 4.5 to about 6.5. Lower pH can be employed. For this fermentation, suitable temperature and pH conditions for Lactobacilli growth are typically from about 30° C. to about 40° C. and a pH from about 5.5 to about 6.2. The broth can be fermented for up to about four days or longer, typically, for about 1 to about 12 hours. The broth can then be pasteurized and packaged. Pasteurization kills remaining live Lactobacillus species in the composition and is optional. Pasteurization can occur via a heat shock of at least 70° C. for 15 min or a sharp change in pH.

The disclosure is also directed to an isolate or a fraction of the resulting keratin fermentation broth. For example, the broth can be subjected to post-fermentation processing, including separating the fermentation biomass (i.e., the Lactobacillus species bacteria) from the remainder of the broth forming a fraction thereof essentially free of particulates, and containing essentially only soluble organic compounds produced by the fermentation and optionally soluble residual substrate ingredients such as sugar should there be any remaining in the broth. Any Lactobacillus species can be removed or separated from the remainder of the broth via filtration or another suitable method to produce a fraction thereof (e.g., the filtrate). Additionally, any individual component of the keratin fermentation broth can be isolated to produce an isolate thereof. For example, this can be isolated keratin and/or hydrolyzed keratin following exposure to fermentation of a simple sugar with the Lactobacillus species. In these and other embodiments, the aqueous keratin fermentation broth can be post-processed to remove water, for example by spray-drying, to yield a powdered organic solid (e.g., an anhydrous fermentation product or a “dried fermentation broth fraction”) that can be rehydrated into an aqueous solution prior to addition to a cosmetic formulation.

The keratin fermentation broth can also include an added preservative. For example, from about 2 to about 50% by weight of a biostatic solvent can be added to the fermentation broth, fraction or isolate thereof to prevent residual Lactobacillus species or other bacteria from growing in an aqueous composition. Suitable biostatic solvents can be selected from the group consisting of glycerin, propanediol, butylene glycol, 1,2-hexanediol, pentylene glycol, and other water-soluble diols.

In some examples, the keratin fermentation broth comprises greater than about 5 wt. %, greater than about 8 wt. %, or greater than about 10 wt. % keratin and/or hydrolyzed keratin, with respect to the total weight of the keratin fermentation broth. In some examples, the keratin fermentation broth comprises greater than about 5 wt. %, greater than about 8 wt. %, or greater than about 10 wt. % hydrolyzed keratin, with respect to the total weight of the keratin fermentation broth. Additionally or alternatively, the keratin fermentation broth may comprise less than about 10 wt. %, e.g., less than about 5 wt. %, less than about 3 wt. %, less than about 2 wt. %, or less than about 1 wt. % of a simple sugar source. The remainder of the keratin fermentation broth typically comprises water or other suitable solvent. In at least one example, the keratin fermentation broth comprises from about 5 wt. % to about 10 wt. % keratin and/or hydrolyzed keratin, from about 0.01 wt. % to about 0.1 wt. % of a Lactobacillus species (e.g., Lactobacillus acidophilius), and from about 0.1 wt. % to about 3 wt. % of a simple sugar source.

### Cosmetic Compositions Comprising Keratin Fermentation Broths

The present disclosure is further directed to cosmetic compositions comprising the keratin fermentation broth described elsewhere in the present application.

The compositions can be formulated in various suitable forms including, for example, low to moderate viscosity liquids, lotions, milks, mousses, sprays, gels, creams, shampoos, conditioners, and the like. In various embodiments, the compositions described herein are formulated as a hair conditioner or shampoo.

In various embodiments, the compositions described herein can have a pH of about 7 or less, about 6.5 or less, about 6 or less, about 5.5 or less, about 5 or less, about 4.5 or less, about 4 or less, or about 3 or less. In some embodiments, the compositions described herein can have a pH of from about 3 to about 7, from about 3 to about 6, from about 3 to about 5, from about 3 to about 4, from about 4 to about 7, from about 4 to about 6, or from about 4 to about 5.

In various embodiments, the compositions described herein can further comprise a solvent. For example, the solvent can comprise an aqueous solvent (e.g., water). In these and other embodiments, the total amount of keratin fermentation broth in the composition is typically at least about 0.05 wt. % and no more than about 20 wt. %. In some embodiments, the total amount of keratin fermentation broth can be about 10 wt. % or less. For example, the total concentration of keratin fermentation broth can be from about 0.05 wt. % to about 10 wt. % or from about 0.1 wt. % to about 10 wt. %. In some embodiments, the total amount of keratin fermentation broth can be about from about 0.05 wt. % to about 3 wt. % or from about 0.1 wt. % to about 3 wt. %. In some particular embodiments, the total amount of keratin fermentation broth can be about 0.05 wt. %, about 0.1 wt. %, about 1 wt. %, about 2 wt. %, or about 3 wt. %.

The compositions described herein may further comprise one or more additives (e.g., cosmetically acceptable ingredients). Examples of cosmetically acceptable ingredients are those listed in the International Cosmetic Ingredient Dictionary and Handbook and those listed in the United States Pharmacopeia. Cosmetically acceptable ingredients include, but are not limited to preservatives, antioxidants, chelating agents, vitamins, dyes, hair coloring agents, proteins, amino acids, natural extracts such as plant extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, absorbents, and combinations thereof.

For example, surfactants include various anionic, cationic, nonionic, and amphoteric surfactants. Anionic surfactants include, but are not limited to, those containing carboxylate, sulfonate and sulfate ions. Examples of anionic surfactants include sodium, potassium, ammonium of long chain alkyl sulfonates and alkyl aryl sulfonates. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

Emollients include, for example, silicone compounds, polyols (e.g., propanediol), and triglycerides.

Emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, polysorbate-80, and combinations thereof.

Preservatives include, but are not limited to, glycerin containing compounds, benzyl alcohol, parabens, sodium benzoate, ethylenediamine-tetraacetic acid (EDTA), potassium sorbate, and so on. Antioxidants include, for example, tocopheryls, BHT, ascorbic acid, Camellia sinensis leaf extract, ascorbyl palmitate, magnesium ascorbyl phosphate, carotenoids, resveratrol, triethyl citrate, arbutin, kojic acid, tetrahexydecyl ascorbate, superoxide dismutase, zinc, sodium metabisulfite, lycopene, ubiquinone, and combinations thereof.

Conditioning agents include, for example, silicone-based agents, panthenol, hydrolyzed wheat and/or soy protein, amino acids, rice bran wax, meadowfoam seed oil, mango seed oil, grape seed oil, jojoba seed oil, sweet almond oil, hydroxyethyl behenamidopropyl diimonium chloride, aloe leaf extract, aloe barbadensis leaf juice, phytantriol, panthenol, retinyl palmitate, behentrimonium methosulfate, cyclopentasiloxane, quaternium-91, stearamidopropyl dimethylamine, and combinations thereof.

Viscosity modifying agents include, for example, viscous liquids, such as polyethylene glycol, semisynthetic polymers, cellulose derivatives, synthetic polymers, naturally occurring polymers, bentonite, colloidal silicon dioxide, and microcrystalline cellulose, and salts, such as sodium chloride, and combinations thereof.

Opacifying agents include, but are not limited to, glycol distearate and ethoxylated fatty alcohols.

In some embodiments, the compositions described herein comprise at least one of a viscosity modifier (e.g., xanthan gum or equivalent), a preservative (e.g., phenoxyethanol), an emollient (e.g., propanediol), a conditioning agent (e.g., stearamidopropyl dimethylamine, behentrimonium methosulfate, and/or sunflower oil), or an emulsifier (e.g., cetearyl alcohol).

### Methods of Use

A further aspect of the present invention is directed to a method of treating hair fibers, the method comprising contacting the hair fibers with a hair treatment composition comprising a keratin fermentation broth comprising a) keratin and/or hydrolyzed keratin, b) Lactobacillus acidophilus, and c) a simple sugar, or a fraction or an isolate thereof.

The hair treatment composition can be any keratin fermentation broth or cosmetic composition described herein.

The hair treatment composition can be formulated as a topical composition including low to moderate viscosity liquids, lotions, milks, mousses, sprays, gels, creams, shampoos, conditioners, and the like.

The hair treatment composition can be applied directly to the hair or scalp, or can be applied as a part of a kit or sequence of hair treatments, especially before, during, or after hair coloring, hair bleaching or hair straightening. Such treatments may be chemical treatments, meaning a treatment with oxidizers and/or reducing chemicals or enzymes which chemically modify the hair or the applied dye. Such hair treatments may be based on application of heat or mechanical force to the hair. The compositions herein may be formulated as leave-in treatments or compositions to be rinsed out after a suitable treatment time.

All percentages are by weight of the total composition unless specifically stated otherwise. When more than one composition is used during a treatment, the total weight to be considered is the total weight of all the compositions applied on hair simultaneously (i.e. the weight found “on head”) unless otherwise specified. All ratios are weight ratios unless specifically stated otherwise.

As used herein the terms “hair” and “hair fibers” to be treated may be “living” (i.e., on a living body) or may be “non-living” (i.e., in a wig, hairpiece or other aggregation of non-living keratinous fibers) and in some embodiments is mammalian hair, particularly human hair. However, wool, fur and other keratin containing fibers are suitable substrates for the compositions according to the present invention.

As used in this application, including the appended claims, the singular forms “a,” “an,” and “the” include plural references unless the content clearly dictates otherwise, and are used interchangeably with “at least one” and “one or more.” Relative terms such as “about,” “substantially,” “approximately,” etc., are used to indicate a possible variation of +10% of a stated numeric value or range.

The invention will be further described in the following examples, which do not limit the scope of the invention described in the claims.

### EXAMPLES

It is to be understood that while the invention has been described in conjunction with the detailed description thereof and accompanying figures, the preceding description is intended to illustrate and not limit the scope of the invention, which is defined by the scope of the appended claims. Other aspects, advantages, and modifications are within the scope of the following claims.

### Example 1: Fermentation Process

To perform the fermentation, 3 kg hydrolyzed wool keratin (from Keraplast (Islington, Christchurch, New Zealand), 10% solution) was added to a glass fermenter and heated to 35° C. Next, 0.4 grams of Lactobacillus acidophilus LA-14 strain (200 billion CFU/g) and 30 grams of edible honey were added. The material was gently mixed and allowed to ferment for 3 hrs. The material was pasteurized and packaged.

Technicians were provided 100 ml jars of CoreTX-Pep™-unfermented hydrolyzed keratin solution from Keraplast and the fermented wool keratin respectively, and asked to compare the odor at room temperature. The CoreTX-Pep™ product was described as having an animal-like odor: words such as barn, horses, and cows were used to describe its odor. On the other hand, the fermented wool keratin was described as having a distinctly more mild odor, with minor animal-like odors, and much more pleasant and tolerable. The drastic change in odor indicates a substantial chemical change in the material.

While soluble wool proteins and peptides are highly advantageous for treating hair, all processed proteins can give rise to unpleasant odors arising from amines and other nitrogen-containing compounds. Amine malodors occur when amino acids in the protein are cleaved and decarboxylated, leaving volatile organic amines of various kinds. Unpleasant amine orders exist in hydrolyzed proteins and peptides of wool. It is important to note that even trace amounts of amine compounds can be unpleasant in cosmetic formulations. All humans have trace amine olfactory receptors that trigger us to be avoidant of undesirable sources of volatile amines (e.g., rotten flesh, degraded food, feces). Therefore, it would be highly advantageous to produce soluble wool peptides having reduced amine odor, and it was a surprising result that the materials of this invention have significantly improved or reduced odor with a distinctly different odor profile and reduced amine malodors as compared to unfermented wool peptides.

### Example 2: Shampoo Formulation

A shampoo formulation comprising the keratin fermentation broth prepared in Example 1 is described in Table 1.

TABLE 1

| Phase | Ingredient | Weight % |
|---|---|---|
| A | Water | QS to 100 |
| | Glycerin | 6 |
| | Guar Gum | 0.8 |
| | Phenoxyethanol | 0.9 |
| | Ethylhexylglycerin | 0.1 |
| B | Decyl Glucoside | 20 |
| C | Cetearyl Alcohol | 4 |
| | MCT Oil | 2 |
| D | Polyquaternium-7 | 2 |
| | Fragrance | QS |
| E | Lactobacillus/Honey/Keratin | 1 |
| | Ferment | |
| F | Citric Acid OR Sodium Hydroxide | QS pH |
| | | 5-5.5 |

This composition is made by slurrying the gum guar in glycerin and adding it to the water phase consisting of the rest of the ingredients of Phase A while stirring. This composition is allowed to hydrate for 10-15 minutes. Then, Phase B is added and stirred under low shear until combined. This is then heated to 75° C. The combined components of Phase C are then added to Phase A/B when at 75° C. and stirred thoroughly under low shear until melted and uniform. After removal from heat, Phase D is added below 40° C., and the result is mixed until uniform. Then Phase E is added and stirred until uniform. The composition is kept below 30° C., and pH is adjusted using Phase F.

### Example 3: Conditioner Formulation

A conditioner formulation comprising the keratin fermentation broth prepared in Example 1 is described in Table 2.

TABLE 2

| Phase | Ingredient | Weight % |
|---|---|---|
| A | Water | QS to 100 |
| | Guar Hydroxypropyltrimonium Chloride | 0.8 |
| B | Behentrimonium Chloride | 2 |
| | Cetearyl Alcohol | 5 |
| | Shea Butter | 2 |
| C | Polyquaternium-7 | 5 |
| | Fragrance | 1 |
| | Vitamin E | 0.5 |
| | Phenoxyethanol | 0.9 |
| | Ethylhexylglycerin | 0.1 |
| D | Lactobacillus/Honey/Keratin | 3 |
| | Ferment | |
| E | Citric Acid OR Sodium Hydroxide | QS pH |
| | | 4-4.5 |

First, the ingredients of Phase 1 are combined, and the guar hydroxypropyltrimonium chloride is dispersed until clump free. This is then heated to 80° C. The combined ingredients of Phase B are then added to Phase A and stirred under high shear until Phase B melts. Stirring is continued while cooling, and at 40° C., Phase C ingredients are added and stirred until uniform. Then, Phase D is added and stirred until uniform. The composition is kept below 30° C., and pH is adjusted using Phase E.

### Example 4: Leave-on Conditioner Formulation

A leave-on condition formulation comprising the keratin fermentation broth prepared in Example 1 is described in Table 3.

TABLE 3

| Phase | Ingredient | Weight % |
|---|---|---|
| A | Water | QS to 100 |
| | Guar Gum | 1 |
| | Propanediol | 5 |
| | Phenoxyethanol | 0.9 |
| | Ethylhexylglycerin | 0.1 |
| B | Cetrimonium Chloride | 1 |
| C | Jojoba Oil | 0.1 |
| | Fragrance | 0.25 |
| D | Lactobacillus/Honey/Keratin | 2 |
| | Ferment | |
| E | Citric Acid OR Sodium Hydroxide | QS pH |
| | | 4-4.5 |

This composition is made by slurrying the guar gum in propanediol and adding it to the water phase consisting of the rest of the ingredients of Phase A while stirring. This composition is allowed to hydrate for 10-15 minutes. Then, Phase B is added and stirred under low shear until combined. Then, Phase C is added and stirred until uniform. Then, Phase D is added and stirred until uniform, and pH is adjusted using Phase E.

### Example 5: Evaluation of the Hair Hydrophobicity

Hair hydrophobicity is an important property of hair that protects hair from excess moisture or humidity. A reduction of hair hydrophobicity from chemical, mechanical, or environmental damage can contribute to hair frizz, tangle, and/or breakage during humid conditions. Thus, there exists a need to restore damaged hair to its more hydrophobic condition. The following example shows that hair treated with a Keratin Fermentation Broth Simple Solution 0.1% treatment had higher hydrophobicity compared to the control.

One type II bleached hair swatch was prepared for each treatment as described below:
1. Control Group (CTR): non-conditioning shampoo 20% SLES (sodium laureth sulfate);
2. Treatment Group (TRT): Keratin Fermentation Broth Simple Solution 0.1%. The ingredients in the Keratin Fermentation Broth Simple Solution 0.1% are shown below in Table 4.

TABLE 4
Keratin Fermentation Broth Simple Solution 0.1% composition

| Phase | Ingredient | Weight % |
|---|---|---|
| A | Water | QS to 100 |
| | Glycerin | 6 |
| | Hydroxyethylcellulose | 1.0 |
| | Polyquaternium-4 | 1.0 |
| B | Cetearyl Alcohol | 5 |
| | Behentrimonium Chloride | 3 |
| | Shea Butter | 2 |
| | Sodium Laneth-40 Malaeta/Styrene | 1 |
| | Sulfonate | |
| C | Lactobacillus/Honey/Keratin | 0.1 |
| | Ferment | |
| D | Phenoxyethanol | 0.9 |
| | Ethylhexylglycerin | 0.1 |
| | Fragrance | QS |

Prior to the application of the treatments, the hair swatches were washed with non-conditioning shampoo to remove any residue the swatches might have. 0.4 mg of 20% SLES per gram of hair swatch was applied. The hair was massaged six times from root to tip end of the hair and rinsed thoroughly with tepid water for 60 seconds.

Keratin Fermentation Broth Simple Solution 0.1%: 0.2 g of the cream per gram of hair was applied and spread on the wet hair. The hair swatch was massaged 6 times from the root to tip.

After applying the test products on the respective hair swatch, they were placed to dry for 24 hours in a chamber with controlled temperature and humidity (22±2° C.; 50±5% RH).

Dried hair swatches were horizontally placed so the fibers were aligned and the water drops were placed in different areas of the hair swatch. The test was filmed for all treatments. The angle of the drop (just after it was deposited over the hair surface) and the remaining time (until the drop was absorbed) were computed to check the formation of a protection film over the hair.

Three contact angle measurements were done for 3 water drops laid onto the hair. The average values of the evaluated parameters were compared by Student t-test (treatment versus control). The level of confidence considered was 95% (α=0.05).

Table 5 presents the statistical results observed in the comparison between the “Keratin Fermentation Broth Simple Solution 0.1%” treatment and control (CTR).

TABLE 5
Descriptive statistics and results of the
comparison between treatment and control

| | Contact Angle (deg) | | Remaining Time (s) | |
|---|---|---|---|---|
| | | Simple | | Simple |
| | | Solution | | Solution |
| Statistics | 20% SLES | 0.1% | 20% SLES | 0.1% |
| | 98.10 | 97.80 | 14.41 | 25.61 |
| | 91.20 | 101.70 | 10.80 | 28.52 |
| | 90.40 | 90.30 | 5.52 | 31.28 |
| Mean | 93.2 | 96.6 | 10.2 | 28.5 |
| Standard | 2.4 | 3.3 | 2.6 | 1.6 |
| Error | | | | |
| 95% CI | [82.7; 103.8] | [82.2; 111] | [−0.9; 21.4] | [21.4; 35.5] |
| Standard | 4.2 | 5.8 | 4.5 | 2.8 |
| Deviation | | | | |
| Median | 91.2 | 97.8 | 10.8 | 28.5 |
| Minimum | 90.4 | 90.3 | 5.5 | 25.6 |
| Maximum | 98.1 | 101.7 | 14.4 | 31.3 |

| | | |
|---|---|---|
| p-value | 0.466 | 0.007* |

*significant at 5% (Student t-test).

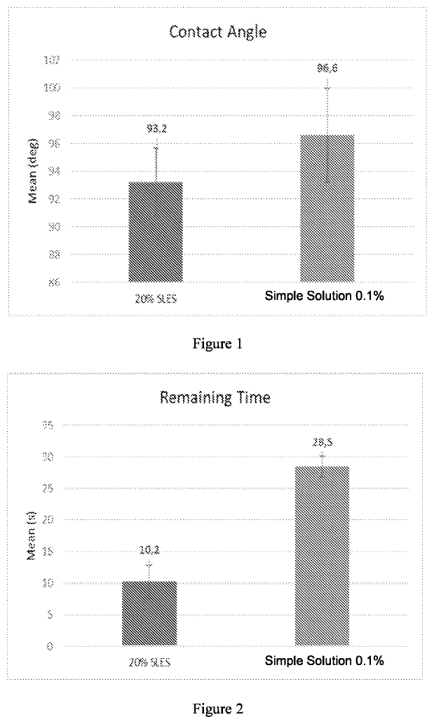

According to the data presented in Table 5 and the statistical comparison performed, it was possible to observe that the control presented no difference in the contact angle compared to the Keratin Fermentation Broth Simple Solution 0.1% treatment (FIG. 1). However, after the Keratin Fermentation Broth Simple Solution 0.1% treatment, the hair took a long time to absorb the water drop, compared to the control (FIG. 2).

Therefore, after the Keratin Fermentation Broth Simple Solution 0.1% treatment, the drop presented a higher remaining time on the hair, and thus a higher hydrophobicity compared to the control.

### Example 6: Breakage Resistance Evaluation

Hair damaged from chemical, mechanical, and environmental causes can become more susceptible to breakage, so there exists a need for hair treatments that help resist hair breakage. The following example shows that a Keratin Fermentation Broth Simple Solution 0.1% treatment presents 59% and 2 times more breakage resistance compared to a control.

Four natural hair swatches type V were prepared for each treatment as described below:
1. Control Group (CTR): non-conditioning shampoo 20% SLES;
2. Treatment Group (TRT): Keratin Fermentation Broth Simple Solution 0.1%. The ingredients in the Keratin Fermentation Broth Simple Solution 0.1% are shown in Table 4.

Before applying the treatments, all hair swatches were washed with non-conditioning shampoo to remove residues. 0.4 ml/g of hair was applied to the hair swatches and spread over the wet hair. The hair swatch was massaged 6 times from the root to tip and rinsed in running water (5±1 L/min and 35±2° C.) for 30 seconds.

The control group swatches were inserted in an automatic brushing machine, BLPA 101, and the test was carried out: 8 cycles of 1000 brushings, counting the fibers at the end of each cycle.

The four remaining hair swatches were treated as follows:

Keratin Fermentation Broth Simple Solution 0.1%: After that, 0.2 g of the cream per gram of hair was applied and spread on the wet hair. The hair swatch was massaged 6 times from the root to the tip.

After the treatment application, the hair swatches were inserted in the automatic brushing machine (BLPA 101) and the test was carried out.

The lower the number of broken fibers, the higher the breakage resistance presented by the treatment will be.

The average values of broken hair fibers were compared by Student's t-test with a confidence interval of 95%.

Equation 1 calculates the breakage resistance, in percentage, compared to the control. Equation 2 calculates how much resistant the hair is after the treatment application compared to the control. These numbers will be presented along with statistical results observed in the following section.

$$\% = ( 1 - TRT / CTR ) \times 100 \qquad Eq . 1$$

$$Number of times = {( TRT / CTR )}^{- 1} \qquad Eq . 2$$

Table 6 presents the number of broken fibers by treatment in each cycle and the statistical results.

TABLE 6
Descriptive statistics per time-point and results
of the comparison between treatments

| Statistics | 20% SLES | Simple Solution 0.1% |
|---|---|---|
| | 31 | 18 |
| | 36 | 18 |
| | 45 | 12 |
| | 42 | 15 |
| | 35 | 12 |
| | 40 | 19 |
| | 42 | 16 |
| | 34 | 16 |
| Sum | 305 | 126 |
| Mean | 38.1 | 15.8 |
| Std. Error | 1.7 | 0.9 |
| 95% CI | [34.1; 42.2] | [13.5; 18] |
| Std. Dev. | 4.8 | 2.7 |
| Median | 38.0 | 16.0 |
| Minimum | 31.0 | 12.0 |
| Maximum | 45.0 | 19.0 |

| | |
|---|---|
| p-value | <0.001* |

*significant at 5% (Student's t-test)

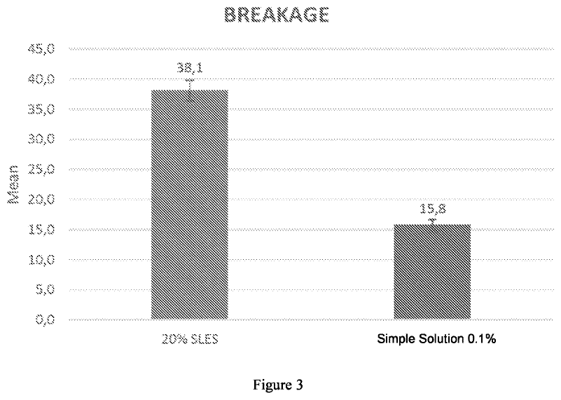

According to the data presented in Table 6 and the statistical comparison performed, it was possible to observe that the Keratin Fermentation Broth Simple Solution 0.1% treatment provided a reduction in hair breakage compared to the control (FIG. 3).

Using equations 1 and 2, it was possible to observe that:
1) After applying the Keratin Fermentation Broth Simple Solution 0.1% treatment the hair was 59% more resistant to breakage;
2) Keratin Fermentation Broth Simple Solution 0.1% treatment presented, approximately, 2 times more resistance than the control.

Thus, it can be concluded that the Keratin Fermentation Broth Simple Solution 0.1% treatment presents 59% and 2 times more breakage resistance compared to the control.

### Example 7: Differential Scanning Calorimetry

Hair integrity, which is a property associated with hair health, can be measured by differential scanning calorimetry (DSC). The following example shows that a Keratin Fermentation Broth Simple Solution 0.1% treatment has increased integrity compared to a control.

One type II bleached hair swatch was selected for each treatment as described below:
1. Control Group (CTR): non-conditioning shampoo 20% SLES;
2. Group TRT1: Keratin Fermentation Broth Simple Solution 0.1%. The ingredients in the Keratin Fermentation Broth Simple Solution 0.1% are shown in Table 4.

Prior to the application of the treatments, the hair swatches were washed with non-conditioning shampoo to remove any residue the swatches might have. 0.4 mg of 20% SLES per gram of hair swatch was applied, the hair was massaged six times from root to tip end of the hair and, then, rinsed thoroughly with tepid water for 60 seconds.

After that, the treatments were applied as follows:

Control: 0.4 mg of 20% SLES per gram of hair was applied, the hair was massaged six times from root to the tip end of the hair and, then, rinsed thoroughly with tepid water for 60 seconds.

Keratin Fermentation Broth Simple Solution 0.1%: 0.2 g of the cream per gram of hair was applied and spread on the wet hair. The hair swatch was massaged 6 times from the root to the tip.

Hair Integrity analysis were done by DSC (differential scanning calorimetry) where the enthalpy of keratin denaturation was obtained and compared between the treatments.

The DSC curves of heat flow and Enthalpy of keratin denaturation were obtained from the treated swatches. Three measurements were done for each treatment and the summary of results can be seen in the next section. The average values of keratin denaturation enthalpy were compared by Student t-test (treatment versus control). A higher enthalpy means more integrity of the hair fiber. The level of confidence considered was 95% (α=0.05).

Equation 3 calculates the percentage compared to the control. Equation 4 calculates the number of times the hair presents after the treatment application compared to the control. These numbers will be presented along with statistical results observed in the following section if the treatment differs from the control group.

$$\% = ( 1 - TRT / CTR ) \times 100 \qquad Eq . 3$$

$$Number of times = {( TRT / CTR )}^{- 1} \qquad Eq . 4$$

Table 7 presents the statistical results observed in the comparison between Keratin Fermentation Broth Simple Solution 0.1% treatment and control (CTR).

Enthalpy (J/g)

| Statistics | CTR | Simple Solution 0.1% |
|---|---|---|
| | 3.00 | 3.57 |
| | 2.77 | 4.65 |
| | 3.10 | 4.41 |
| Mean | 2.96 | 4.21 |
| Standard Error | 0.10 | 0.33 |
| 95% CI | 0.17 | 0.57 |
| Standard Deviation | 3.00 | 4.41 |
| Median | 2.77 | 3.57 |
| Minimum | 3.10 | 4.65 |
| Maximum | 2.96 | 4.21 |

| | |
|---|---|
| p-value | 0.021* |

*significant at 5% (Student t-test)

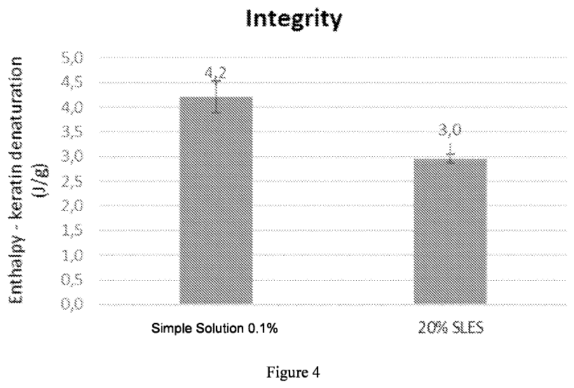

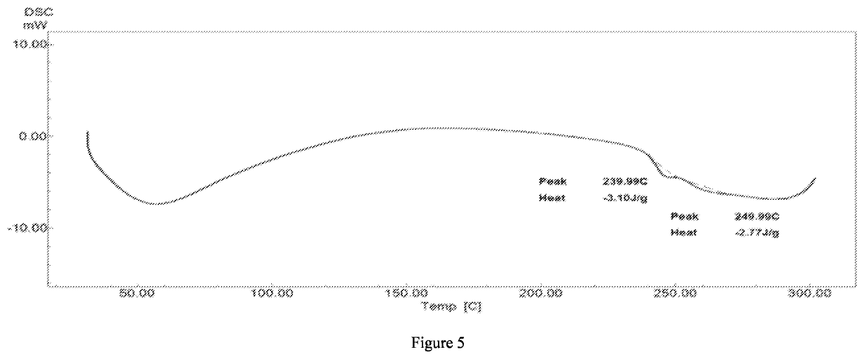

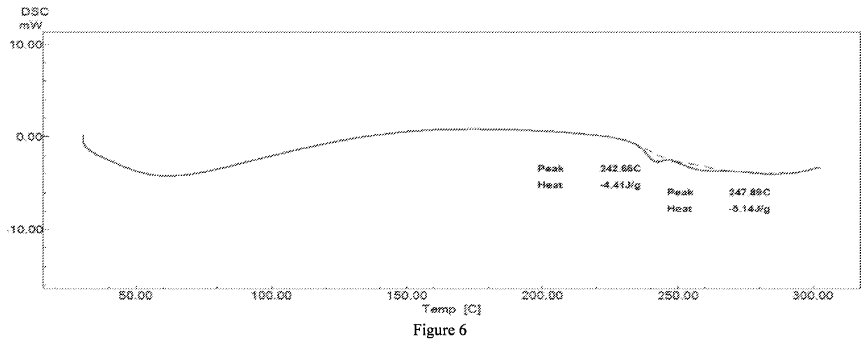

According to the data presented in Table 7 and the statistical comparison performed, the Keratin Fermentation Broth Simple Solution 0.1% treatment presented an enthalpy of keratin denaturation approximately 42% higher than the control (FIG. 4-6).

Therefore, the Keratin Fermentation Broth Simple Solution 0.1% treatment presented higher integrity compared to the control.

### Example 8: Luster Evaluation

Lustrous hair is a highly desired quality of consumers and generally indicate that hair is in good condition. The following examples shows that the Keratin Fermentation Broth Simple Solution 0.1% treatment restores luster to hair compared to a negative control when both hair samples are dirtied with artificial sebum.

As described below, six natural type I swatches were separated for each treatment.
1. Positive Control Group (CTRP): non-conditioning shampoo (20% SLES).
2. Treatment Group (TRT): Keratin Fermentation Broth Simple Solution 0.1%. The ingredients in the Keratin Fermentation Broth Simple Solution 0.1% are shown in Table 4.

The swatches initially received the application of artificial sebum (Negative Control Group-CTRN), so all of them presented the same dirt condition.

After the dirtying process, the swatches received their respective application, as described below:

CTRP: 4 ml/g of hair was applied to the swatches and spread on wet hair. Each hair swatch was massaged 6 times from the root to the tip, and rinsed in running water (5±1 L/min and 35±2° C.) for 30 seconds.

Keratin Fermentation Broth Simple Solution 0.1%: After that, 0.2 g of the cream per gram of hair was applied and spread on the wet hair. Each hair swatch was massaged 6 times from the root to the tip.

After applying the treatments, the swatches were photographed in a luster chamber. The images were analyzed via ImageJ®, and the light intensity was quantified for each of the images.

From the quantification of the light intensity profile, the specular and diffuse reflections were calculated and used to calculate the luster according to equation 5:

$${B}_{ALL} = ( {D}_{Stamm} / {S}_{Gaussian} ) \times ( {I}_{MAX} / {w}_{1 / 2} ) \qquad Eq . 5$$

Where B_{ALL} is the luster data internally developed to correlate with the sensorial perception of the luster from the images acquired within the luster chamber (Internal development); Dₛₜₐₘₘ is diffuse reflection proposed by Stamm and Fuchs from the light intensity profile; S_{Gaussian} is the first specular reflection; IMAX is the maximum intensity of the profile; and w_{1/2} is the width at half height of the intensity profile (or the luminous bandwidth in the image). For each swatch, the luster was calculated and the mean value was compared between non-conditioning shampoo and the treatment with fortifying shampoo and conditioner.

Equation 6 calculates the percentage compared to the non-conditioning shampoo. Equation 7 calculates the number of times the hair presents after the treatment application compared to the non-conditioning shampoo. These numbers will be presented along with statistical results observed in the following section if the treatment differs from the control group.

$$\% = ( 1 - TRT / CTR ) \times 100 \qquad Eq . 6$$

$$Number of times = {( TRT / CTR )}^{- 1} \qquad Eq . 7$$

The next section presents the observed results.

Table 8 presents the statistical results observed in the comparison between the treatment and control groups.

TABLE 8
Descriptive statistics per time-point and results
of the comparison between treatments

| | | | Simple |
|---|---|---|---|
| Statistic | CTRP | CTRN | Solution 0.1% |
| | 6.65 | 3.91 | 5.74 |
| | 5.63 | 3.71 | 6.55 |
| | 5.98 | 3.74 | 5.73 |
| | 7.63 | 3.72 | 5.95 |
| | 6.83 | 3.92 | 7.19 |
| | 6.53 | 3.69 | 7.07 |
| Mean | 6.54 | 3.78 | 6.37 |
| Standard Error | 0.28 | 0.04 | 0.27 |
| 95% CI | [5.81; 7.27] | [3.67; 3.89] | [5.68; 7.06] |
| Standard | 0.70 | 0.10 | 0.66 |
| Deviation | | | |
| Median | 6.59 | 3.73 | 6.25 |
| Minimum | 5.63 | 3.69 | 5.73 |
| Maximum | 7.63 | 3.92 | 7.19 |

TABLE 9
Statistical comparison and p-values

| Contrast | Mean Difference | P-value |
|---|---|---|
| Simple Solution 0.1% vs | 2.588 | <0.001* |
| CTRN | | |
| Simple Solution 0.1% vs | 0.171 | 0.858 |
| CTRP | | |
| CTRP vs CTRN | 2.759 | <0.001* |

*Significant at 5% level (ANOVA - Tukey test)

According to the data presented in Table 8 and the statistical comparison presented in Table 9, it was possible to observe that:
1) The Keratin Fermentation Broth Simple Solution 0.1% treatment presented approximately 69% more luster than the negative control (CTRN);
2) The Keratin Fermentation Broth Simple Solution 0.1% treatment shows no significant difference compared to the non-conditioning shampoo (CTRP).

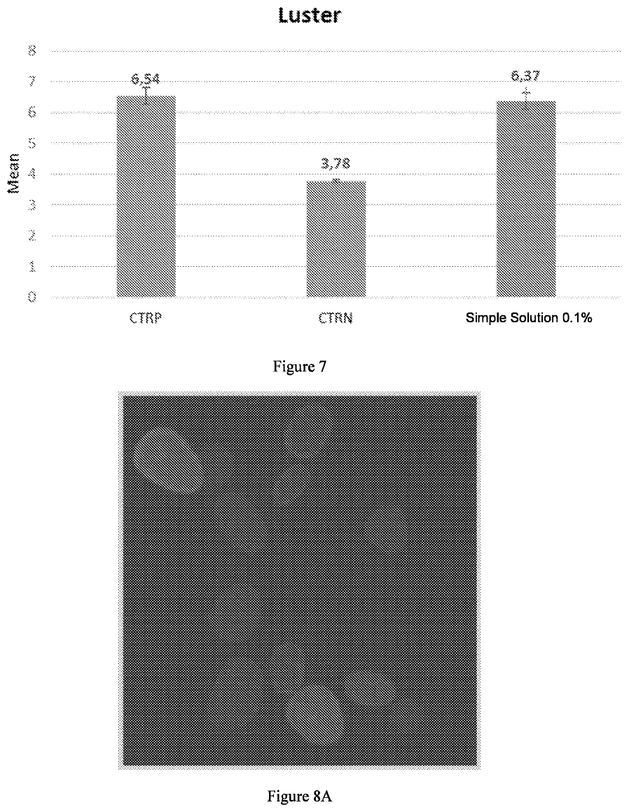

There was a significant increase in luster compared to the Negative Control group, and there was no difference between the Keratin Fermentation Broth Simple Solution 0.1% treatment and Positive Control group. Therefore, we can conclude that the Keratin Fermentation Broth Simple Solution 0.1% product restores luster to the hair (FIG. 7).

### Example 9: Visualize the Penetration of Specific Peptide into Hair Fibers Using Fluorescence Microscopy

Hair is formed from layers of polymerized keratin protein, and keratin-containing cosmetic formulations are thought to strengthen hair by fortifying and repairing keratin structure. However, the success of these formulations requires keratin that is able to penetrate into hair fibers.

### Key Points

The hydrolyzed keratin peptides in the Fermented Binding Serum were successfully labeled with a fluorescence probe and their penetration was visualized into relaxed hair fibers.

The Fermented Binding Serum peptide penetrated the whole cortex of relaxed hair fibers.

The penetration of Fermented Binding Serum peptides into the cortex of relaxed hair fibers was significantly higher compared to the penetration of higher molecular weight peptides (Keratec™; Croda Inc.).

The Keratec™ peptides (high MW; Croda Inc.) accumulated only at the cuticle and can reach in rare cases the most external part of the cortex in some regions of relaxed hair fibers, likely where the cuticle is the most damaged.

### Materials and Methods

Substrate: 10 mg of relaxed hair fibers (~30 hair fibers).

### Products:

Fermented Binding Serum (Actera Ingredients; Lot #0462), ~4,500 Da MW). This material was prepared in accordance with the process described in Example 1.

Keratec™ IFP PE (Croda Inc.; Batch #22A26B105), ~50,000 Da MW.

### Samples:

Control relaxed hair fibers-treated with purified dye-only control solution.

Relaxed hair fibers treated with labeled Fermented Binding Serum.

Relaxed hair fibers treated with labeled Keratec™.

### Sample Preparation:

1. Both products were dialyzed against phosphate buffer solution (PBS) for at least 16 hours using a dialysis cassette and a floating device; the molecular weight cut-off (MWCO) of the cassettes was 2,000 Da. This step ensures the removal of amine-containing substances that could interfere with the fluorescent labeling.
a. 10 mL of peptide solutions are injected into pre-wetted cassettes (Slide-A-Lyzer, Thermo Scientific) following the manufacturer's protocol.
b. The dialysis cassettes were immersed in 2 L of fresh PBS using a floating device for 2 hours at room temperature with gentle stirring of the buffer.
c. The buffer was replaced with 2 L of fresh PBS and dialyzed for another 2 hours at room temperature.
d. The buffer is replaced with fresh PBS again and placed in the fridge to dialyze for 16 hours at 4° C.

2. The dialyzed peptide solutions are evaluated for protein concentration using BCA assay (ThermoFisher Scientific).
a. Protein concentration in the dialyzed peptide solutions is determined against a standard curve of known protein concentrations.

3. A volume of peptide solutions corresponding to ~10 mg of protein was used to carry out the fluorescence labeling reaction.

4. Optimal fluorescence labeling requires a solution with slightly basic pH; thus, 1M sodium bicarbonate buffer was utilized to adjust the pH of solutions.

5. 5(6)-Carboxytetramethylrhodamine N-succinimidyl ester (Sigma Aldrich) was used for fluorescence labeling of the peptides. This amine reactive Rhodamine dye has an approximate excitation/emission maxima of ~546/579 nm, which helps avoid autofluorescence of hair fibers which can be observed at 350-450 nm.

6. A dye working solution was prepared in DMSO at 10 mg/mL.

7. A volume corresponding to 1 mg of dye was slowly added to the dialyzed peptide solutions with continuous stirring. A dye-only control was prepared in a similar fashion by adding 1 mg of dye to sodium bicarbonate buffer (without peptides).

8. The conjugation reaction was carried out at room temperature with continuous shaking for 1 hour.

9. The labeled peptide solutions were further purified by centrifugal devices with 1,000 MWCO for Fermented Binding Serum peptide solutions and 10,000 MWCO for Keratec™ peptide solutions. This step removes any unbound dye present in the solution and concentrates the labeled peptides.
a. 5 mL of labeled peptide solutions were loaded onto respective MWCO centrifugal devices (Microsep™ Advance Centrifugal Devices with Omega membrane, Fisher Scientific).
b. The centrifugal devices were centrifuged at 6,500×g at 24° C. with a spin time of 20 minutes. This step was repeated 15 times using 1×PBS for washing after every spin cycle. After 15 times, the flow-through was visibly clear.
c. Purified labeled peptide solutions were re-suspended in 10 mL of fresh 1×PBS and used for soaking treatments and penetration evaluation.

10. ~ 30 relaxed MB hair fibers were bundled and immersed in 1.5 mL of labeled peptide solutions (0.1% labeled peptide solutions).

11. The hair samples were incubated at room temperature with gentle shaking for 24 hours.

12. After 24 hours, the hair bundles were removed from the solutions, rinsed for 20 seconds with DI water and dried with a blow dryer on cool setting for 10 seconds.

13. Hair fibers were prepared for cryosectioning, and 10 μm cross-sections were collected for imaging with light and fluorescence microscopy.

### Microscopy of Treated Hair Fibers: Fluorescence Labeled Peptide

Brightfield microscopy and fluorescence images were collected with an Eclipse TE2000-U microscope (Nikon Instruments Inc.) equipped with a Kiralux compact scientific camera (ThorLabs Inc.), Prior Lumen 200 Fluorescence Illumination System and a Nikon DS-Qi2 monochrome camera. 15-20 hair cross-sections (10 μm) were analyzed at 20× and 100× magnifications. Fluorescence images were acquired in the red channel (CY3 filter; Excitation: 560/40 nm; Emission: 630/75 nm) at 20× and 100× magnifications, with 10 ms and 5 ms exposures time respectively for the red channel (CY3), and 50 ms in the green channel using NIS Elements software. The natural fluorescence of the hair fibers was recorded in the green channel, while the labeled peptides emit fluorescence in the red channel.

In addition to fluorescence microscopy, semi-quantitative total fluorescence analyses were conducted. This data was reported in bar graphs.

### Results

Following dialysis, the BCA protein quantification assay was used to determine the concentration of peptides against a standard curve of known concentrations. This step was performed prior to fluorescence labeling to calculate the volume required for 10 mg of “protein” for proper controlled labeling.

### Control Results

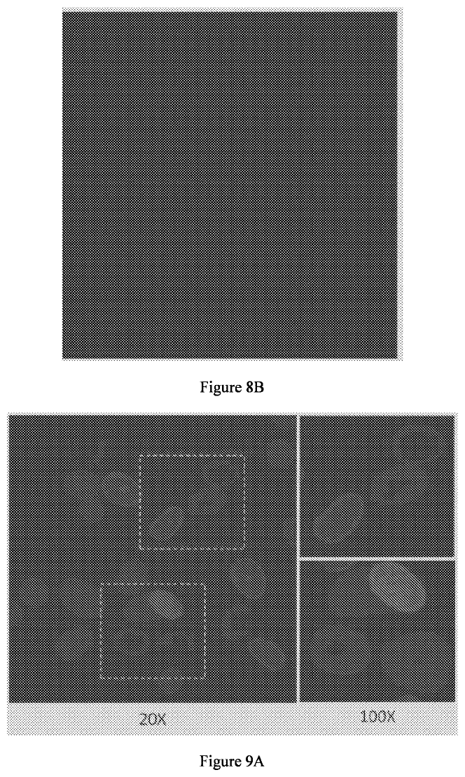

Representative images for each sample and treatment are presented in this report, and the images were collected with the same parameters for all the groups. In this experiment, a dye-only control was purified in parallel to the labeled peptide solutions and evaluated for penetration. As anticipated, no fluorescence was detected in the red channel from the dye-only control after centrifugal purification using the centrifugal column devices (FIG. 8A-8B). This result confirms the effectiveness of the washing protocol to remove free/unbound dye from the sample solution.

### Fermented Binding Serum Penetration Results

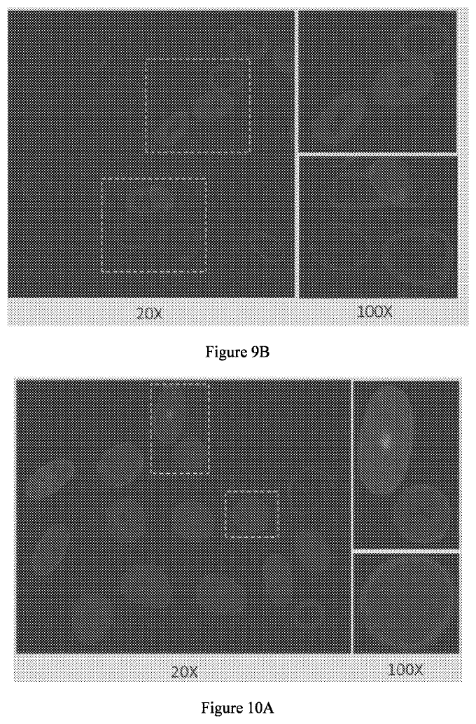

After confirmation that no free dye remains in the solution following the washing protocol, fluorescence in the red channel can be exclusively attributed to the labeled peptide and a direct assessment of peptide penetration inside the hair fibers. The Fermented Binding Serum peptide penetrated the whole cortex of relaxed hair fibers; a strong and uniform fluorescence intensity is observed across all hair cross-sections evaluated in this project and the peptide can be seen all inside the cortex (FIG. 9A-9B).

### Keratec™ Penetration Results

Unlike the Fermented Binding Serum, Keratec™ concentrated only at the cuticle of the hair fibers, and in 1-2 cross-sections, minimal penetration into the most external regions of the cortex could be observed (FIG. 10A-10B). The manufacturer specified that Keratec™ contains hydrolyzed keratin peptides in the molecular weight range of 40,000 to 60,000 Daltons. However, due to the manufacturing process, Croda Inc. warns about the presence of peptides in the 3,000 to 4,000 Daltons weight range. The centrifugal device selected to purify labeled Keratec™ had a 10,000 Da molecular weight cut-off to alleviate this potential discrepancy. Labeled peptides above 10,000 Da MW would be concentrated, while anything below should be washed out.

The substrate used was relaxed hair, which means that the cuticle can become damaged from the chemical treatment. A possible explanation for the regions of external cortex penetration by this high MW peptide is that the cuticle was damaged enough in those regions to allow some penetration of Keratec™.

Total Fluorescence Analysis as Semi-Quantitative Assessment of Peptide Penetration into Hair Fibers

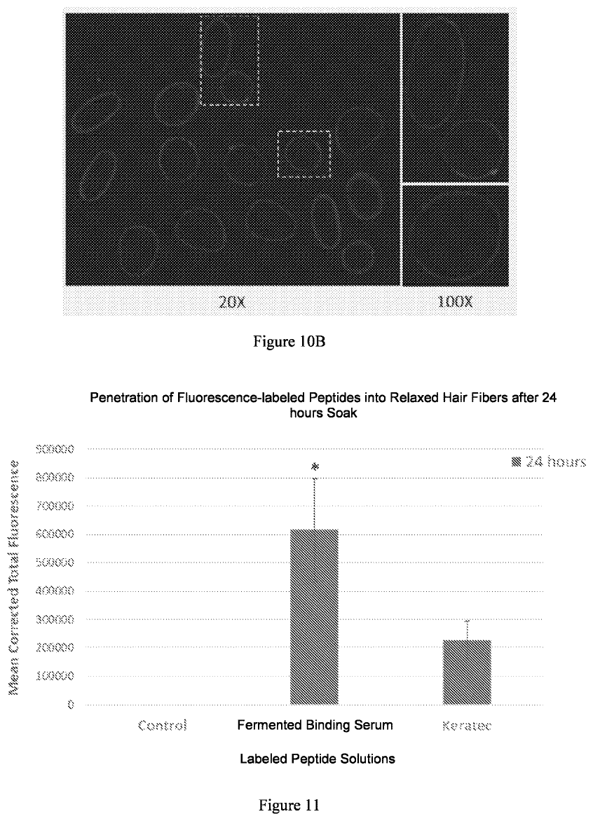

FIG. 11 shows a semi-quantitative analysis of total fluorescence intensity of the hair cross-sections after treatment with labeled peptide for 24 hours. The values were corrected for the image background. The results are reported in mean±standard deviation. The control refers to the hair soaked for 24 hours in dye-only solution after purification. The corrected total fluorescence value corresponding to the penetration of the Fermented Binding Serum is significantly higher compared to that of Keratec™, further supporting the increased penetration of the Fermented Binding Serum peptides into the hair cortex seen after 24 hours of treatment.

TABLE 10
Mean corrected total fluorescence

| | Corrected Total | |
|---|---|---|
| Labeled Peptides | Fluorescence | p-value |
| Control | 78.2 | |
| Fermented Binding Serum | 616,406.7 | 0.000583 |
| Keratec ™ | 227,017.8 | |

### Conclusions

The hydrolyzed keratin peptides in the Fermented Binding Serum were successfully labeled with a fluorescence probe and their penetration was visualized into relaxed hair fibers.

The Fermented Binding Serum peptide penetrated the whole cortex of relaxed hair fibers.

The penetration of Fermented Binding Serum peptides into the cortex of relaxed hair fibers was by far significantly higher compared to the penetration of higher molecular weight peptides (Keratec™; Croda Inc.).

The Keratec™ peptides (high MW; Croda Inc.) accumulated only at the cuticle and can reach in rare cases the most external part of the cortex in some regions of relaxed hair fibers, likely where the cuticle is the most damaged.

### Example 10: Odor Analysis by Means of VDI3882 Tests

### Methodology

### Sample Preparation for Sensory Analysis (VDI3882 and Character)

Half milliliter (0.5 ml) of each sample (CoreTX-Pep™-unfermented hydrolyzed keratin control or Fermented Binding Serum-fermented hydrolyzed keratin prepared in accordance with the process described in Example 1) was placed in a 250 ml vial and heated at 35° C. for 24 hours to promote the release of odorants. The samples were then presented to the panelists for sensory evaluation.

For the evaluation test of odor intensity, hedonic tone and character, the panelists evaluated the sample directly at room temperature. The samples were presented blindly to the individuals, in triplicates, resulting in each panelist assessing three different pieces of the same sample.

### Odor Intensity, Hedonic Tone and Character Analysis

The intensity, hedonic tone (property related to the pleasantness or unpleasantness of an odor), and character of samples were analyzed. A total of 6 individuals constituted the panel members. They were calibrated according to the UNE-EN 13725:2004 “Air Quality—Determination of odor concentration by dynamic olfactometry” normative and trained based on the German standard VDI3882.

The assessment of both intensity and hedonic tone parameters was performed independently, with the objective of determining the potential of an annoyance of a certain odor. These parameters were evaluated based on predefined scales, as established in the VDI3882 normative. For the intensity parameter, the scale of assessment varies between the value 0 (not perceptible) to the value 6 (extremely strong), the intermediate value of 3 represents an intensity that is distinguishable (Table 11).

TABLE 11
Odor intensity scale according to VDI3882

| Odor intensity | Rating scale |
|---|---|
| Extremely strong | 6 |
| Very strong | 5 |
| Strong | 4 |
| Distinct | 3 |
| Weak | 2 |
| Very weak | 1 |
| Not detectable | 0 |

In relation to hedonic tone, the scale goes from the negative value of −4 (extremely unpleasant) to the positive value of +4 (extremely pleasant), the value of 0 represents the situation where the smell is neither pleasant nor unpleasant (Table 12). This classification allows a direct comparison of the samples under study, by inhaling directly the odor emitted by each.

TABLE 12
Odor hedonic tone according to VDI3882

| Hedonic tone | Rating scale |
|---|---|
| Extremely pleasant | +4 |
| Very pleasant | +3 |
| Pleasant | +2 |
| Slightly pleasant | +1 |
| Neutral | 0 |
| Slightly unpleasant | −1 |
| Unpleasant | −2 |
| Very unpleasant | −3 |
| Extremely unpleasant | −4 |

These tests determined if the odors emitted by the different samples generated an odor perception considered with a relevant/low intensity and/or (un) pleasantness by the panel members. Moreover, these tests allowed the definition of the characters inherent to the samples and the identification of similarities or differences that can be related to the VOCs determined by GC-Sniffing-MS.

### Results

### Sensory Evaluation of the Odor Intensity and Hedonic Tone

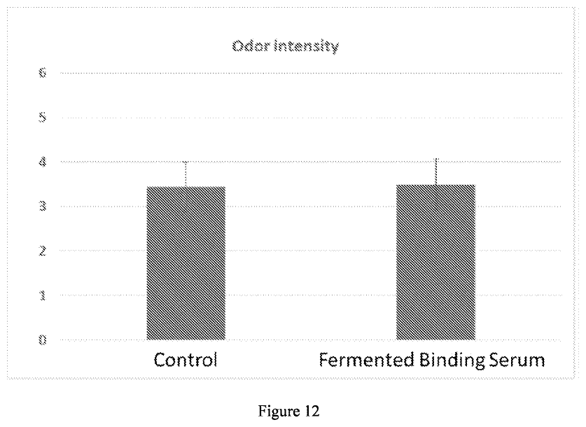

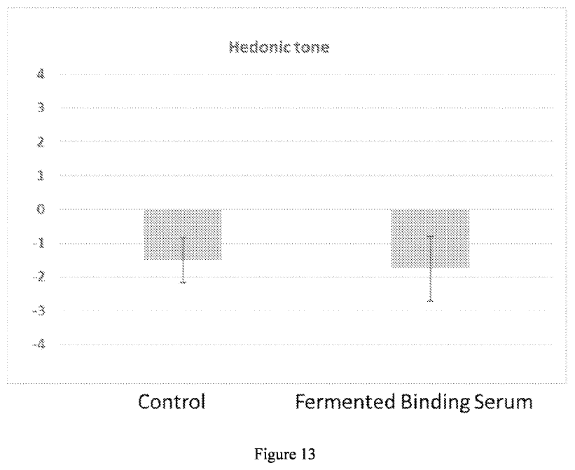

Assessment of the odor strength (intensity) and level of pleasantness/unpleasantness (hedonic tone) of each sample, by direct sniffing, was carried out by group of 6 calibrated and trained panel members. This assay was performed based on the German normative VDI3882. Additionally, to the referred measurements, a description of the odor character generated by sample was also carried out by the same panel members during the assay. Panelists were provided with a list of each sample's odor descriptors related to sample characteristics and as a guide. The assessments were performed in triplicate. The summary of these results is presented in Table 13 and displayed in FIGS. 12 and 13.

TABLE 13
Summary on the sensory evaluation concerning odor
intensity and hedonic tone. Average and standard
deviations on panelists' scores are presented.

| | | Fermented |
|---|---|---|
| Parameter | Control | Binding Serum |
| Odor intensity | | |
| Average | 3.44 | 3.50 |
| Standard deviation | 0.38 | 0.42 |
| Hedonic tone | | |
| Average | −1.50 | −1.75 |
| Standard deviation | 0.79 | 0.39 |
| Character | | |
| 1st most reported | rancid/putrid (22) | animal (21) |
| 2nd most reported | pungent/acid (18) | hay/dry herb (15) |
| 3^{rd} most reported | toasty/cereals (16) | phenolic (15) |

The odor intensities of both samples following heating at 35° C. were comparable, with score close to 3.5, “distinct-strong”. As for the hedonic tone, both samples had a value close to −1.5 (standard deviation smaller than 1), being ‘slightly unpleasant-unpleasant with notable differences in odor attributes. The main odor notes in the unfermented hydrolyzed keratin control samples were described as rancid/putrid, pungent/acid, and toasty/cereal, while the main odor notes in the Fermented Binding Serum were described as animal, phenolic, hay/dry herb.

T-student test was used for evaluating the statistical significance of paired comparisons between the samples, in relation to the scores provided by the sensory panel. P-values were determined and used for hypothesis evaluation (n<20); samples were accepted to be significantly different at α=0.05 (two tailed). Differences in intensity and hedonic tone between the samples when heated to 35° C. were not statistically significant (p-value for intensity 0.16; p-value hedonic tone 0.18).

The t-student tests revealed that there are not significant differences between the samples for both intensity and hedonic tone (p>0.05).

### General Perception of Odors

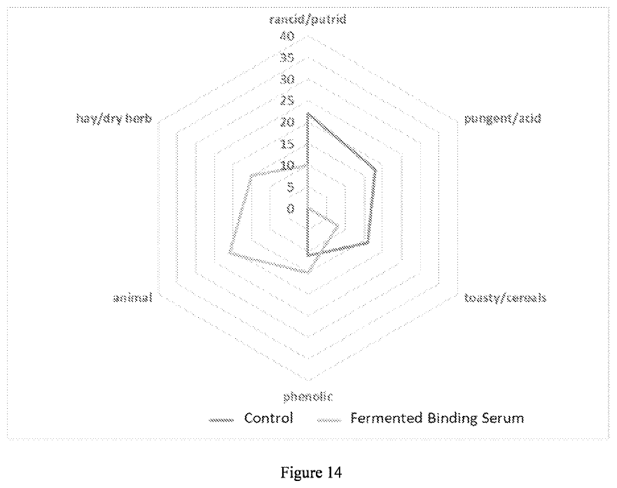

To reach a better representation of the information, the individual odors perceived during the analyses were grouped according to a more general description, obtaining in this way 6 new categories. A general value of intensity was obtained by adding up of the values of intensity of all odors belonging to a new category. The summary of this information is shown in Table 14. Table 14 represents an attempt to simplify the sensory information obtained in each sample to provide an assessment of the general sensory profile, and note differences between samples and the sensory character where the differences become more significant. FIG. 14 represents graphically (spider chart) the information presented in table 14.

TABLE 14
Odors classified by general categories. Intensity
value represents the total sum of individual odors.

Sum of odor intensities

| Category | Control | Fermented Binding Serum |
|---|---|---|
| rancid/putrid | 22 | 10 |
| pungent/acid | 18 | 0 |
| toasty/cereals | 16 | 8 |
| phenolic | 11 | 15 |
| animal | 0 | 21 |
| hay/dry herb | 0 | 15 |

The most prominent odor categories were distinctly different between the two samples, with rancid/putrid, pungent/acid, and toasty/cereal being reported for the unfermented hydrolyzed keratin control and animal, phenolic, hay/dry herb being reported for the Fermented Binding Serum.

### Example 11: Gc-ToF-Ms Testing

### Methodology

### Sample Preparation for GC-ToF-MS Analysis

Sample preparation for the high-resolution GC-ToF-MS analysis consisted of adding 2 ml of sample material (CoreTX-Pep™-unfermented hydrolyzed keratin control or Fermented Binding Serum-fermented hydrolyzed keratin) and introducing it in separate microchambers of 114 ml capacity each. Once closed, the microchamber conditions were adjusted to 35° C., where the respective samples were kept promoting the emission of volatiles at this temperature. In this way, the application conditions of the samples in the final products were simulated. Then, two adsorption tubes (Tenax/carbograph) were fitted to the upper lid of each microchamber, where the volatiles emitted by each sample were collected by a volume of 1000 ml per sample of gas within the tubes. Afterwards, the tubes were hermetically closed with caps.

### High Resolution GC-ToF-MS Analysis

After the collection of VOCs into adsorption tubes, the tubes were inserted into the thermal desorption unit coupled to GC-(TOF)MS. The instrumentation system consisted of a gas chromatograph (Agilent 7890, USA), a mass spectrometer with time-of-flight analyzer (BenchTOF-dx, Almsco, Germany), and a thermal desorption unit (Unity2, Markes, UK). The desorbed amount of the tube, according to a specific temperature program, was optimized to ensure good resolution of the analysis.

After being removed from the tube by thermal desorption, volatile compounds were captured in a cold trap at a low temperature (−20° C. to 10° C.) by thermoelectric cooling. Subsequently, the cold trap was heated to 300° C.-350° C. according to a programmed temperature profile, to release all volatiles up to the GC for subsequent chromatographic separation. At the end of the GC column, once separated, the compounds reached the mass detector with different retention times, where they were ionized and, through the selector Flight Time (Time-of-flight, TOF), the mass of ions was determined with high precision to enable identification.

Data provided by each sample was then analyzed. This analysis was done partly automatically, using advanced software and compound libraries. System calibration was performed in-house using a wide range of compounds. The analyses of this study were performed semi-quantitatively, using as reference solution Toluene-d8, to prioritize a full scan quantification of all VOCs present in the samples.

### Results

### High Resolution GC-ToF-MS Analysis

The unfermented hydrolyzed keratin control exhibited nearly twice the number of VOCs as the Fermented Binding Serum. In total, 67 VOCs were identified in the samples tested: unfermented hydrolyzed keratin control (CoreTX-Pep™) (55); fermented hydrolyzed keratin (Fermented Binding Serum) (28).

### Comparative VOCs Composition Per Chemical Group

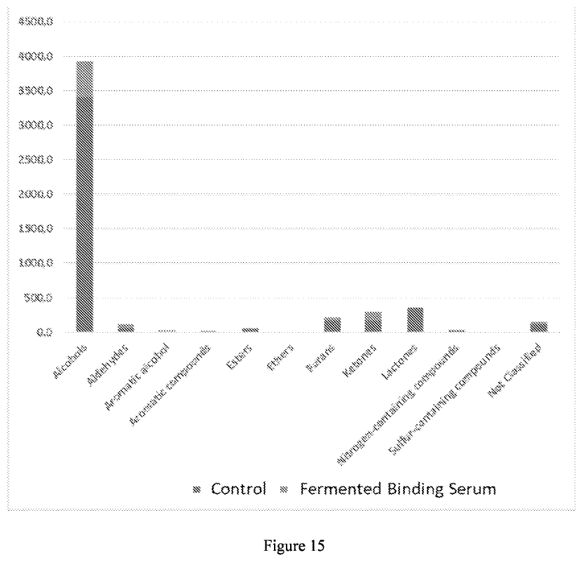

Table 15 shows the summarized information of VOC concentrations measured in the samples based on the total concentration per chemical groups of compounds. The information provided by Table 15 is represented graphically in FIG. 15. The concentration of total VOCs in the unfermented hydrolyzed keratin control (4378.6 μg/m³) was higher than in the Fermented Binding Serum sample (824.4 μg/m³). The predominant family in the samples was alcohols, comprising 77% in unfermented hydrolyzed keratin control and 64% in the Fermented Binding Serum. Some of the predominant alcohols included ethanol, 2-phenoxy-(control), isopropyl alcohol, and linalool (Fermented Binding Serum).

As indicated above in Example 1, even trace amounts of amine compounds can be unpleasant in cosmetic formulations. It was a surprising result that the Fermented Binding Serum had no nitrogen-containing compounds detected while the unfermented hydrolyzed keratin control had 32.0 g/m³.

TABLE 15
Results of total VOCs (μg/m³) in each
sample classified by chemical groups

Concentration (μg/m³)

| | | Fermented |
|---|---|---|
| Chemical groups | Control | Binding Serum |
| Alcohols | 3396.7 | 529.8 |
| Aldehydes | 58.8 | 59.4 |
| Aromatic alcohol | 11.9 | 14.5 |
| Aromatic compounds | 11.8 | 6.0 |
| Esters | 52.1 | 5.5 |
| Ethers | 0.0 | 0.0 |
| Furans | 170.2 | 44.8 |
| Ketones | 175.7 | 127.0 |
| Lactones | 357.4 | 0.0 |
| Nitrogen-containing | 32.0 | 0.0 |
| compounds | | |
| Sulfur-containing | 0.0 | 8.7 |
| compounds | | |
| Not Classified | 112.0 | 28.8 |
| Total VOCs | 4378.6 | 824.4 |

### Relevant Odorants Identified by GC-ToF-MS

To understand the “weight” of the perception of an odorant, the Odor Activity Value (OAV) is typically used. This is calculated from the abundance of that odorant molecule (concentration) divided by its Odor Threshold Value (OTV). The odor threshold value is understood as the lowest concentration of a particular odorous compound that is detectable by the human sense of smell. In practice, this value is variable, as human olfactory sensitivity varies widely. This uncertainty has been addressed by making OTVs traceable to an agreed-upon reference stimulus, as outlined in the European standard for olfactometry EN13725:2003.

The compounds with the highest OAV values for the unfermented hydrolyzed keratin control were for phenol, 2-methoxy- and butanal, 3-methyl- with OAV values considered distinguishable. These compounds are typically understood to be associated with odor notes of aldehydic, fatty, nutty, phenolic, and medicine. In contrast, the most relevant compounds for the Fermented Binding Serum sample were linalool and butanal, 3-methyl-, with OAV values considered distinguishable. These compounds are typically understood to be associated with odor notes of aldehydic, fatty, bergamot, and floral (Table 16).

TABLE 16
Relevant odor-active compounds and their odor activity value (OAV)

| | | Concentration | | Odor active value | | |
|---|---|---|---|---|---|---|
| | | (μg/m³) | | (OAV) | | |
| | | | Fermented | | Fermented | |
| | CAS | | Binding | | Binding | Odor |
| Compound | No. | Control | Serum | Control | Serum | descriptor |
| Propanal, 2- | 78-84- | 3.30 | 2.25 | 3* | 2* | fresh |
| methyl- | 2 | | | | | aldehydic |
| | | | | | | floral green |
| Butanal, 3- | 590- | 11.97 | 10.60 | 20** | 18** | etheral |
| methyl- | 86-3 | | | | | aldehydic |
| | | | | | | chocolate |
| | | | | | | peach fatty |
| Benzaldehyde | 100- | 21.34 | 37.22 | 1* | 2* | strong sharp |
| | 52-7 | | | | | sweet bitter |
| | | | | | | almond |
| | | | | | | cherry |
| Decanal | 112- | 10.49 | <0.01 | 4* | <1 | sweet |
| | 31-2 | | | | | aldehydic |
| | | | | | | waxy orange |
| | | | | | | peel citrus |
| | | | | | | floral |
| Phenol, 2- | 90-05- | 11.87 | <0.01 | 32** | <1 | burnt, |
| methoxy- | 1 | | | | | medicine, |
| | | | | | | phenol, |
| | | | | | | smoke, |
| | | | | | | wood |
| Benzoic acid, | 93-58- | 3.30 | <0.01 | 1* | <1 | phenolic |
| methyl ester | 3 | | | | | wintergreen |
| | | | | | | almond |
| | | | | | | floral |
| Linalool | 78-70- | <0.01 | 124.84 | <1 | 67** | bergamot, |
| | 6 | | | | | coriander, |
| | | | | | | floral, |
| | | | | | | flower, |
| | | | | | | grape |
| Acetaldehyde | 75-07- | <0.01 | 5.69 | <1 | 2* | pungent |
| | 0 | | | | | ethereal |
| | | | | | | aldehydic |
| | | | | | | fruity |
| Octanal | 124- | <0.01 | 0.78 | <1 | 15* | aldehydic |
| | 13-0 | | | | | waxy citrus |
| | | | | | | orange peel |
| | | | | | | green fatty |
| Eucalyptol | 470- | <0.01 | 8.29 | <1 | 1* | camphor, |
| | 82-6 | | | | | cool, mint, |
| | | | | | | sweet |

For Table 16, the concentrations in bold exceed the odor threshold value (OTV). The concentrations in italics did not exceed 0.01 μg/m³. Values indicated by * (1 to 15) indicate very weak, barely recognizable odors. Values indicated by ** (15 to 500) indicate distinct, easily recognizable but not strong odors. Values of 500 to 5000 indicate a moderate to strong odor. Values of 5000 to 50,000 indicate a very strong odor. Values greater than 50,000 indicate an extremely strong odor. The concentration of acetaldehyde could not be determined accurately.

1. A keratin fermentation broth comprising
a) keratin and/or hydrolyzed keratin,
b) a Lactobacillus species, and
c) a simple sugar
or a fraction or an isolate thereof.

2. The keratin fermentation broth of claim 1, wherein the simple sugar is provided by honey and the keratin fermentation broth comprises less than about 5 wt % of the simple sugar.

3. The keratin fermentation broth of claim 1, wherein the keratin and/or hydrolyzed keratin is extracted from wool and the keratin fermentation broth comprises greater than about 5 wt. % keratin and/or hydrolyzed keratin.

4. The keratin fermentation broth of claim 1, wherein the Lactobacillus species is Lactobacillus acidophilus.

5. (canceled)

6. The keratin fermentation broth of claim 1, wherein the pH is from about 4 to about 7.

7. The keratin fermentation broth of claim 1, wherein the keratin fermentation broth comprises an aqueous fermentation broth.

8. The keratin fermentation broth of claim 1, wherein the keratin fermentation broth comprises a dried fermentation broth fraction.

9. The keratin fermentation broth of claim 1, wherein the keratin and/or hydrolyzed keratin has an average molecular weight of less than 10 kDa.

10. The keratin fermentation broth of claim 1, wherein the keratin and/or hydrolyzed keratin has an average molecular weight of about 2.5 kDa or about 4.5 kDa.

11. A cosmetic composition comprising the keratin fermentation broth of claim 1, or a fraction or an isolate thereof.

12. The cosmetic composition of claim 11, wherein the composition is formulated as a lotion, milk, mousse, spray, gel, cream, shampoo, or conditioner.

15. The cosmetic composition of claim 11, wherein the concentration of the keratin fermentation broth or a fraction or an isolate thereof is about 0.05 wt. % to about 10 wt. %.

17. The cosmetic composition of claim 11, wherein the concentration of the keratin fermentation broth or a fraction or an isolate thereof is about 0.05 wt. % to about 3 wt. %.

18. (canceled)

19. (canceled)

20. (canceled)

21. (canceled)

22. The cosmetic composition of claim 11, wherein the composition further comprises at least one additive selected from the group consisting of surfactants, vitamins, natural extracts, preservatives, chelating agents, perfumes, preservatives, antioxidants, proteins, amino acids, humectants, fragrances, emollients, penetrants, thickeners, viscosity modifiers, hair fixatives, film formers, emulsifiers, opacifying agents, propellants, liquid vehicles, carriers, salts, pH adjusting agents, neutralizing agents, buffers, hair conditioning agents, anti-static agents, anti-frizz agents, anti-dandruff agents, and combinations thereof.

23. A method of treating hair fibers, the method comprising contacting the hair fibers with the cosmetic composition of claim 11.

24. (canceled)

25. A method of producing a keratin fermentation broth, the method comprising:
a) combining keratin and/or hydrolyzed keratin, Lactobacillus acidophilus, and a simple sugar in an aqueous fermentation solution; and
b) fermenting the aqueous fermentation solution at a temperature of from about 2° C. to about 53° C. and a pH of from about 4.5 to about 6.5 to produce the keratin fermentation broth.

26. The method of claim 25, wherein the aqueous fermentation solution is fermented for about 1 to about 12 hours.

27. The method of claim 25, wherein step b) is performed at a temperature of from about 30° C. to about 40° C.

28. The method of claim 25, wherein step b) is performed at pH of from about 5.5 to about 6.2.

29. (canceled)

30. A product comprising keratin and/or hydrolyzed keratin exposed to fermentation of a simple sugar with a Lactobacillus species.
